(19) United States
(12) Patent Application Publication
(10) Pub. No.: US 2024/0127482 A1
(43) Pub. Date: Apr. 18, 2024

(54) IMAGING OF ULTRASOUND FIELDS USING CALIBRATION FOR NON-LINEAR MAGNETIC FIELD GRADIENT

(71) Applicant: Brigham Young University (BYU), Provo, UT (US)

(72) Inventors: Steven Paul Allen, Springville, UT (US); Davi Ferrazza Cavinatto, Provo, UT (US); Evan Samuel Conger, Provo, UT (US); Paul-Emile Victor Passe-Carlus, Rexburg, ID (US)

(21) Appl. No.: 18/486,779

(22) Filed: Oct. 13, 2023

Related U.S. Application Data
(60) Provisional application No. 63/379,444, filed on Oct. 13, 2022.

(51) Int. Cl. G06T 7/80

(52) U.S. Cl. CPC G06T 7/80; G06T 2207/10088; G06T 2207/10132

(57) ABSTRACT

A method includes determining, during a calibration phase without application of ultrasound waves, a per-pixel magnetic field gradient of a magnetic field that is generated using a calibration drive signal applied to a magnetic coil; generating, during a treatment phase, a magnetic field gradient that is applied to a treatment area by using a treatment drive signal applied to the magnetic coil, wherein treatment drive signal is different from the calibration drive signal; determining, based on the treatment drive signal applied to the magnetic coil, per-pixel phases of a treatment image of the treatment area, wherein the per-pixel phases of the treatment image are based on spin displacement at each pixel resulting from the applied ultrasound waves; and adjusting, for each pixel of the treatment image, the per-pixel phases of the treatment image based on the per-pixel magnetic field gradient for a corresponding pixel, to obtain a gradient-adjusted treatment image.

### CROSS-REFERENCE TO RELATED APPLICATIONS

This application claims priority to U.S. Provisional Application No. 63/379,444, filed on Oct. 13, 2022, entitled, “QUANTIFICATION OF ACOUSTIC PRESSURE WAVES TRAVERSING THROUGH A BODY USING AN ELECTROMAGNET IN CONJUNCTION WITH A MAGNETIC RESONANCE IMAGING SYSTEM AND AN ULTRASONIC TRANSDUCER”, the entirety of which is hereby incorporated by reference.

### TECHNICAL FIELD

This description relates to magnetic resonance imaging (MRI) and imaging of ultrasound fields.

### BACKGROUND

Many people suffer disability, and approximately one third of whom suffer cognitive disabilities or pathology in the central nervous system (CNS). Therapeutic ultrasound is a promising technology that may be used to improve neuron repair and regeneration. The technology includes transmitting acoustic (ultrasound) waves from an ultrasound transducer, through overlying tissue, and down to the brain or spinal parenchyma.

### SUMMARY

According to an example embodiment, a method may include determining, during a calibration phase without application of ultrasound waves, a per-pixel magnetic field gradient of a magnetic field that is generated using a calibration drive signal applied to a magnetic coil; generating, during a treatment phase, a magnetic field gradient that is applied to a treatment area by using a treatment drive signal applied to the magnetic coil, wherein the treatment drive signal is different from the calibration drive signal; determining, based on the treatment drive signal applied to the magnetic coil, per-pixel phases of a treatment image of the treatment area, wherein the per-pixel phases of the treatment image are based on spin displacement at each pixel resulting from the applied ultrasound waves; and adjusting, for each pixel of the treatment image, the per-pixel phases of the treatment image based on the per-pixel magnetic field gradient for a corresponding pixel, to obtain a gradient-adjusted treatment image.

According to an example embodiment, an apparatus may include at least one processor, and at least one memory including computer program code, the at least one memory and the computer program code configured to, with the at least one processor, cause the apparatus at least to determine, during a calibration phase without application of ultrasound waves, a per-pixel magnetic field gradient of a magnetic field that is generated using a calibration drive signal applied to a magnetic coil; generate, during a treatment phase, a magnetic field gradient that is applied to a treatment area by using a treatment drive signal applied to the magnetic coil, wherein the treatment drive signal is different from the calibration drive signal; determine, based on the treatment drive signal applied to the magnetic coil, per-pixel phases of a treatment image of the treatment area, wherein the per-pixel phases of the treatment image are based on spin displacement at each pixel resulting from the applied ultrasound waves; and adjust, for each pixel of the treatment image, the per-pixel phases of the treatment image based on the per-pixel magnetic field gradient for a corresponding pixel, to obtain a gradient-adjusted treatment image.

According to an example embodiment, an apparatus may include a non-transitory computer-readable storage medium comprising instructions stored thereon that, when executed by at least one processor, are configured to cause a computing system to determine, during a calibration phase without application of ultrasound waves, a per-pixel magnetic field gradient of a magnetic field that is generated using a calibration drive signal applied to a magnetic coil; generate, during a treatment phase, a magnetic field gradient that is applied to a treatment area by using a treatment drive signal applied to the magnetic coil, wherein the treatment drive signal is different from the calibration drive signal; determine, based on the treatment drive signal applied to the magnetic coil, per-pixel phases of a treatment image of the treatment area, wherein the per-pixel phases of the treatment image are based on spin displacement at each pixel resulting from the applied ultrasound waves; and adjust, for each pixel of the treatment image, the per-pixel phases of the treatment image based on the per-pixel magnetic field gradient for a corresponding pixel, to obtain a gradient-adjusted treatment image.

Other example embodiments are provided or described for each of the example methods, including: means for performing any of the example methods; a non-transitory computer-readable storage medium comprising instructions stored thereon that, when executed by at least one processor, are configured to cause a computing system to perform any of the example methods; and an apparatus including at least one processor, and at least one memory including computer program code, the at least one memory and the computer program code configured to, with the at least one processor, cause the apparatus at least to perform any of the example methods.

The details of one or more examples of embodiments are set forth in the accompanying drawings and the description below. Other features will be apparent from the description and drawings, and from the claims.

### BRIEF DESCRIPTION OF THE DRAWINGS

FIG. 1 is a block diagram of a magnetic resonance imaging (MRI) system in which therapeutic ultrasound may be used to treat a patient according to an example embodiment.

FIG. 2 is a diagram of example magnetic coils that may be used to generate magnetic fields according to example embodiments.

FIG. 3 is a diagram illustrating a magnetic field gradient as a function of distance from the coil.

FIG. 4 is a diagram illustrating operation of a system that may operate in a calibration phase and in a treatment phase according to an example embodiment.

FIG. 5 is a diagram illustrating a damped sinusoid, which is an example waveform for the calibration drive signal used to drive the magnetic coil during calibration.

FIG. 6 is a flow chart illustrating operation of a system according to an example embodiment.

FIG. 7 is a flow chart illustrating operation of a system according to another example embodiment.

### DETAILED DESCRIPTION

Therapeutic ultrasound's noninvasive nature, focal delivery, and ability to treat many targets within the cranium and spine render it highly compatible for therapy delivery to spatially select targets.

Ultrasound is a powerful therapeutic tool that may be used to resolve movement disorders and shows significant promise toward treating CNS tumors, mood disorders, hematoma, and other pathologies. In focused ultrasound neuromodulation, beams of ultrasonic energy are transmitted through a patient's skull and into their brain. The acoustic vibrations then interact with and modify neurological processes at a desired target in a desired manner to produce a desired medical outcome. Neurological processes are sensitive to the amplitude, frequency, and other parameters associated with the transmitting acoustic vibrations. However, it is very difficult to predict exactly what those properties will be at the desired target in the brain because the human skull modifies the propagating acoustic pulses in complex ways that are hard to predict. Challenges exist in accurately quantifying parameters associated with ultrasound treatment processes.

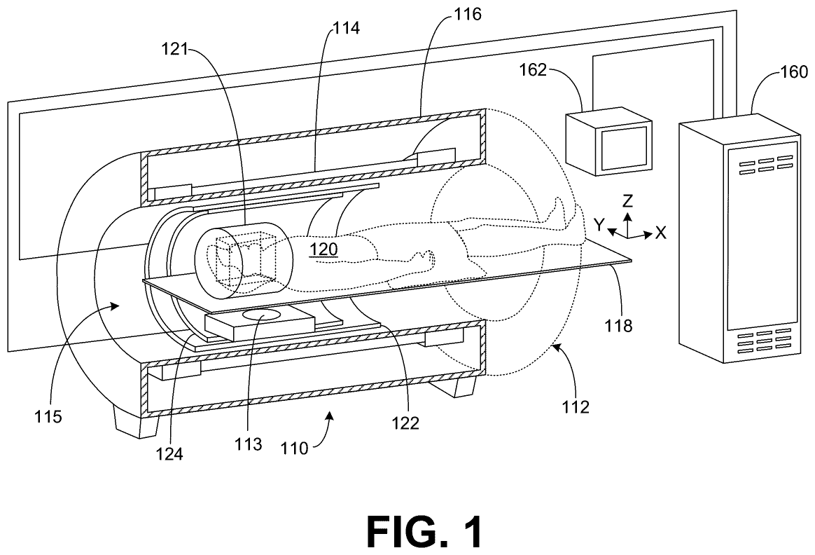

FIG. 1 is a block diagram of a magnetic resonance imaging (MRI) system in which therapeutic ultrasound may be used to treat a patient.

System 110 is shown, and may include a MRI system 112. During treatment an ultrasound transducer 113 may be disposed within the bore of the MRI system 112. The ultrasound transducer may produce ultrasound waves that may be applied to a region of a patient, such as a brain or spine. The MRI system 112 may include a cylindrical electromagnet 114, or other types of electromagnet, which may generate a (e.g., static) magnetic field within a bore 115 of the electromagnet 114. The electromagnet 114 may be enclosed in a magnet housing 116. A support table 118, upon which patient 120 lies during treatment, may be located within the magnet bore 105. Patient 120 is positioned such that an imaging region 121(e.g., the patient's brain, internal organ, or other tissue to receive ultrasound treatment may receive both energy, such as, ultrasound waves from thermal therapy device 103 (e.g., from an ultrasound transducer) and receive the magnetic field.

The MRI system 112 may include a set of cylindrical magnetic field gradient coils 112, which may be located within the magnet bore 115, surrounding the patient 120. The gradient coils 122 can generate magnetic field gradients, which are assumed to be linear. One or more gradient coils 122 may be provided, wherein each gradient coil (or magnetic coil) may generate magnetic field gradients in mutually orthogonal directions. Using the field gradients, different spatial locations can be associated with different precession frequencies, thereby giving an MR image its spatial resolution. Further, an RF transmitter coil 124 surrounds the imaging region 121. The RF transmitter coil 124 emits an RF excitation pulse (or multiple RF excitation pulses) into the imaging region 116, thereby changing the net magnetization of the imaged tissue. The RF transmitter coil 124 (which may include one or more coils) may also be used to receive MR response signals emitted from the imaging region 121. The MR response signals are amplified, conditioned, digitized into raw data, and converted into arrays of image data using an processing system 160. The image data may then be displayed on a monitor 162, which may be any display.

In some example MR imaging procedures, the emission of the RF excitation pulse(s), the application of the field gradients in one or more directions, and the acquisition of the RF response signal may be performed in a predetermined sequence. For example, in some imaging sequences, a field gradient or magnetic gradient (which is typically linear) may be applied parallel to the static magnetic field and applied simultaneously with the RF excitation pulse to select a slice within the three-dimensional tissue for imaging. Subsequently, time-dependent gradients parallel to the imaging plane may be used to impart a position-dependent phase and frequency on the magnetization vector. Alternatively, an imaging sequence may be designed for a three-dimensional imaging region.

The time-varying RF response signal, which may be integrated over the entire (two- or three-dimensional) imaging region, may be sampled to produce a time series of response signals that constitute the raw image data, e.g., a treatment image of the imaging region. Each data point in this time series can be interpreted as the value of the Fourier transform of the position-dependent local magnetization at a particular point in k space, where k is a function of the time development of the gradient fields. Thus, by acquiring a time series of the response signal and Fourier-transforming it, a real-space image of the tissue (e.g., an image showing the measured magnetization-affecting tissue properties as a function of spatial coordinates) can be reconstructed from the raw data. Computational methods for constructing real-space image data from the raw data (including, e.g., Fast Fourier Transform) may be performed by processing system 160 in hardware, software, or a combination of both.

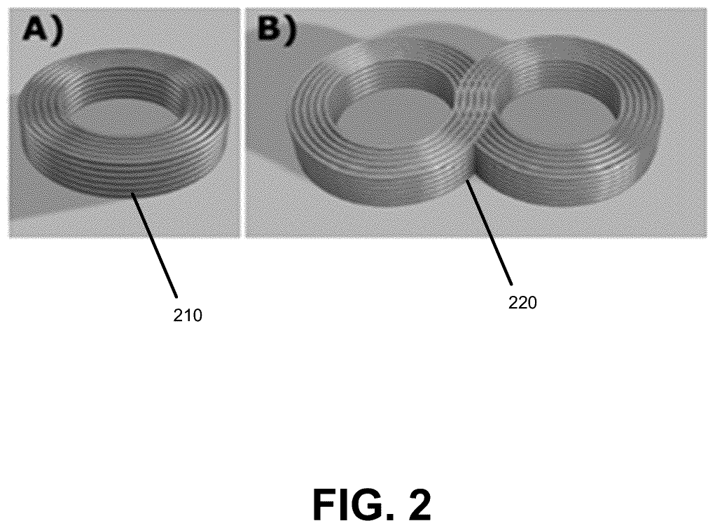

FIG. 2 is a diagram of example magnetic coils that may be used to generate magnetic fields according to example embodiments. Magnetic coils 210 and 220 may be inserted into or provided within an MRI system 112 (including a MRI scanner) that can encode acoustic longitudinal displacement fields into images of living subjects. This displacement data can then be used to determine or estimate acoustic parameters of tissue or a treatment area, or to determine or estimate physical properties of the tissue or treatment area, such as bulk modulus.

Magnetic coil 210 is a single coil design, while magnetic coil 220 is a figure-eight design. In some embodiments, coils 210 and 220 may have a form factor that may be smaller than other magnetic coils so that the magnetic coil is suitable for use in modern MRI scanners, and/or may be used on human anatomy (such as to apply a magnetic field to a human head). As an illustrative example embodiment, a single coil design, such as shown by magnetic coil 210, may include a 3 cm coil driven with 20A of current through 20 windings, which may generate a magnetic field having a magnetic field gradient. Magnetic field gradient is a slope or first spatial derivative of the magnetic field with respect to distance from the magnetic coil. The single coil design of magnetic coil 210 is a simpler design, while the figure-eight design of magnetic coil 220 may exhibit higher magnetic field strengths at greater depths of the brain or other treatment area.

Magnetic field gradient linearity may be an important assumption for quantifying acoustic longitudinal displacement. Typical magnetic coils for MRI systems may have a sufficient size and/or number of windings to generate a linear magnetic field gradient within the expected treatment area.

In some cases, magnetic coils, such as magnetic coils 210 and/or 220 may have a design or structure that causes the magnetic coil to generate a non-linear magnetic field gradient. For example, in some example embodiments, smaller magnetic coils, e.g., smaller diameter coils and/or coils having fewer windings, may have a smaller form factor that is more suitable for MRI systems, have less power consumption, and/or have a size that more suitably fits the physical constraints of human subjects (such as more easily applied to the human head), and/or may be more suitable for a modern MRI system.

Thus, for example, magnetic coil 210, which may use fewer windings, may provide a smaller form factor, use less power, and be more suitable for human anatomy and modern MRI systems, at the cost of (or having a disadvantage of) generating a non-linear magnetic field gradient over a treatment area.

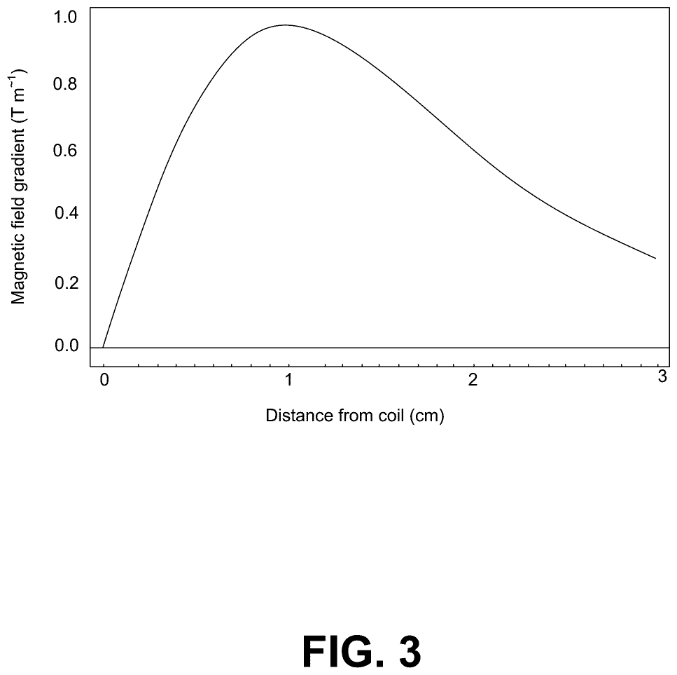

FIG. 3 is a diagram illustrating a magnetic field gradient as a function of distance from the coil. The Y or vertical axis of FIG. 2 is magnetic field gradient (in Teslas per meter), and the X or horizontal axis is distance from the coil (in cm). If an example treatment area is 0.5 cm to 3 cm from the magnetic coil, it can be seen that the magnetic field gradient shown in FIG. 2 is non-linear. The magnetic field gradient increases and/or has an upward or positive slope from 0 cm to about 1 cm (e.g., within a first region of the treatment area that includes the first 1 cm of the treatment area), and then has a negative or downward slope from about 1 cm beyond 3 cm (e.g., within a second region of the treatment area). Also, for example, the magnetic field gradient from 1 cm to 3 cm is not linear, but flattens out (has less slope) at about 2.5 to 3 cm. Thus, these are a few examples indicating that the magnetic field gradient shown in the example of FIG. 2 is non-linear.

As noted above, at least in some cases for MRI systems, it may be desirable or advantageous to use magnetic coils that have a design or structure that may generate non-linear magnetic field gradients. However, an image generated based on a non-linear magnetic field gradient may generate phase information on a MRI image that is inaccurate or in error, due to the non-linearity of the magnetic field gradient (e.g., since linearity of the magnetic field gradient may be assumed during processing of the MRI image). These inaccuracies or errors in the MRI image, resulting from the non-linear magnetic field gradient, may cause problems for surgeons that rely on such MRI images during therapeutic ultrasound or other treatments.

Therefore, according to an example embodiment, techniques are provided in which a per-pixel magnetic field gradient is determined for a magnetic coil during a calibration phase (without ultrasound waves being applied). During a treatment phase (in which ultrasound waves are applied), per-pixel phases are determined for a treatment image. The per-pixel phases of the treatment image are then adjusted or scaled based on the per-pixel magnetic field gradient obtained during the calibration phase.

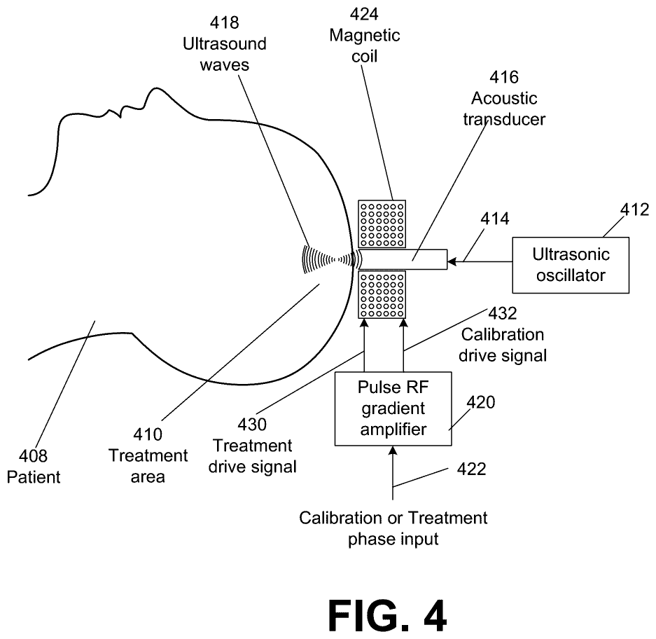

FIG. 4 is a diagram illustrating operation of a system that may operate in a calibration phase and in a treatment phase according to an example embodiment. A patient 408 may receive ultrasound therapy at a treatment area 410. A MRI system 112 (FIG. 1) may be used to perform imaging during the ultrasound therapy, e.g., to generate one or more treatment images.

Referring to FIG. 4, a pulse RF (radio frequency) gradient amplifier 420 may apply or output a drive signal (of multiple possible drive signals) to a magnetic coil 424, to cause magnetic coil 424 to generate a magnetic field having a magnetic field gradient. A phase (or mode) input signal is input via line 422 to pulse RF gradient amplifier 420, indicating either a calibration phase or a treatment phase. Calibration phase may refer to or may include calibration of the magnetic coil 424, including determining of a per-pixel magnetic field gradient. Treatment phase may refer to or include a period of time when ultrasound waves 418 are applied to treatment area 410.

Pulse RF gradient amplifier 420 outputs a calibration drive signal via line 432 to magnetic coil 424 when the phase input signal received via line 422 indicates calibration phase (or during the calibration phase). Pulse RF gradient amplifier 420 outputs a treatment drive signal via line 430 to magnetic coil 424 when the phase input signal received via line 422 indicates treatment phase (or during treatment phase). The treatment drive signal may be different from the calibration drive signal. No ultrasound waves are applied to the treatment area 410 during calibration phase. Ultrasound waves 418 and a magnetic field gradient are applied to the treatment area 410 during treatment phase.

During the calibration phase (without application of ultrasound waves, or when ultrasound waves are not applied to the treatment area 410), the processing system 160 (FIG. 1) may determine, based on a calibration image received by a MRI scanner of the MRI system 112, a per-pixel magnetic field gradient of a magnetic field that is generated using the calibration drive signal applied via line 432 to magnetic coil 424.

During the treatment phase, ultrasonic oscillator 412 outputs a drive signal via line 414 to acoustic transducer 416 to cause the acoustic transducer 416 to generate or apply ultrasound waves 418 to the treatment area 410. Also, during the treatment phase, a magnetic field gradient is generated and applied to treatment area 410 by using (or based on the application of) a treatment drive signal applied via line 430 to the magnetic coil 424.

Also, during treatment phase, processing system 160 (FIG. 1) determines, based on the treatment drive signal applied to the magnetic coil (which results in the magnetic field applied to the treatment area 410), per-pixel phases of a treatment image of the treatment area 410, wherein the per-pixel phases of the treatment image are based on spin displacement at each pixel resulting from the applied ultrasound waves.

According to an example embodiment, the treatment drive signal (applied to the magnetic coil 424 during treatment phase via line 430) may be different from the calibration drive signal (applied to the magnetic coil 424 during calibration phase via line 432). Also, for example, the calibration drive signal may have a waveform with a non-zero time-integral. Also, for example, the treatment drive signal may have a waveform type that matches a waveform type of ultrasound waves that are applied to the treatment area during the treatment phase. Also, for example, the treatment drive signal (and the signal drive signal applied to the acoustic transducer) may have a waveform with a zero time-integral, e.g., such as a sinusoid.

Thus, in an example embodiment, during treatment phase, the drive signals applied to both the magnetic coil 424 and the acoustic transducer 416 may be sinusoids (which have a zero time-integral). And, during calibration phase, a calibration drive signal may be applied to the magnetic coil 424, where the calibration drive signal has a different waveform type than the treatment drive signal (e.g., the treatment drive signal may be a sinusoid (or other waveform) that has a zero time-integral, and the calibration drive signal may be another type of waveform that has a non-zero time-integral). The basis or advantages of using different signals and/or different waveform types for the calibration drive signal and the treatment drive signal are briefly described below.

During treatment phase: Ultrasound waves applied to the treatment area 410 cause the particle spins to oscillate. The oscillation of the magnetic field should be synchronized (e.g., same frequency and/or same waveform type) with the oscillation of the spins that result from the ultrasound waves. Thus, during the treatment phase, the treatment drive signal that is applied via line 430 to the magnetic coil 424, and the drive signal that is applied via line 414 to the acoustic transducer 416, should have the same frequency (e.g., 500 Khz) and the same waveform type (e.g., a sinusoid), e.g., to allow the magnetic field to impart to spins undergoing oscillation phase proportional to the degree of their motion. A sinusoid has a zero time-integral (e.g., because upper (or positive) lobes and lower (or negative) lobes of a sinusoid over time are balanced or equal). During treatment phase, a waveform type (e.g., sinusoid) of the treatment drive signal that matches a waveform type (e.g., sinusoid) of the ultrasound drive signal allows the pixels, when undergoing ultrasound oscillatory movement, to experience a magnetic field that appears to them in their oscillatory state to have a non-zero time integral.

During calibration phase: There are no ultrasound waves applied to the treatment area 410 to cause the particle spins to oscillate. During calibration phase, a different (or different type of) drive signal (the calibration drive signal, which is different than the treatment drive signal) is applied to the magnetic coil 424 to cause the particle spins to accrue phase (because there is no ultrasound waves applied during calibration phase). This different drive signal (the calibration drive signal) applied to the magnetic coil 424 during calibration may cause the particle spins to accrue phase, e.g., which may be used in the absence of the ultrasound waves during calibration. A calibration drive signal with a non-zero time integral allows the pixels to accrue a non-zero phase during image acquisition.

Thus, for example, different drive signals for the magnetic coil 424 may be used or applied during calibration phase and treatment phase. In an example embodiment, the calibration drive signal may have a waveform with a non-zero time-integral. Also, for example, the treatment drive signal may have a frequency (e.g., 500 Khz) and waveform type (e.g., sinusoid) that matches a frequency and waveform type of ultrasound waves 418 that are applied to the treatment area 410 during the treatment phase, e.g., to allow imaging of the treatment area to be performed by the MRI system 112 during treatment.

According to an example embodiment, during calibration phase, waveforms that may be used as calibration drive signals applied to the magnetic coil 424 to cause particle spins to accrue phase during calibration may include waveforms that have a non-zero time integral. Waveforms that have a non-zero time integral cause particle spins to accrue phase because, e.g., of an imbalance of the upper (positive) lobes compared to the lower (negative) lobes of the signal. Some examples of waveforms that have a non-zero time integral may include, e.g., an exponential waveform, a trapezoidal waveform, a square waveform, a ramp waveform, a triangle waveform, and/or a damped sinusoidal waveform.

Also, the processing system 160 may adjust the per-pixel phases of the treatment image (determined during the treatment phase) based on the per-pixel magnetic field gradient (determined during calibration phase) for a corresponding pixel, to obtain a gradient-adjusted treatment image, e.g., which may include a gradient-adjusted per-pixel phase for each pixel of the treatment image. For example, the adjusting may include scaling, for each pixel of the treatment image, the per-pixel phases of the treatment image by or based on the per-pixel magnetic field gradient for the corresponding pixel, to obtain the gradient-adjusted treatment image. Scaling, for example, may include multiplying or dividing the per pixel phases (obtained during treatment phase) by the per-pixel magnetic field gradient (obtained during calibration phase) for the corresponding pixel. In this manner, a non-linear magnetic field gradient may be compensated for in the treatment image, which may allow a wider range of magnetic coils (e.g., which may have a smaller form factor, a fewer number of windings, . . . ) to be used, while still providing accurate imaging during treatment.

After the gradient-adjusted per-pixel phase has been determined or obtained for each pixel of the gradient-adjusted treatment image, the processing system 160 may determine or calculate, per-pixel, at least one acoustic parameter for the treatment area 410 (or for the treatment image) based on the gradient-adjusted per-pixel phase of the gradient-adjusted treatment image. Examples of acoustic parameters that may be determined or calculated may include: acoustic pressure amplitude; sound speed; or acoustic intensity. Other acoustic parameters may be determined or calculated.

In addition, after the gradient-adjusted per-pixel phase has been determined or obtained, the processing system 160 may determine or calculate, per-pixel, a bulk modulus for the treatment area 410 based on the gradient-adjusted per-pixel phase of the gradient-adjusted treatment image. Bulk modulus may indicate a stiffness of the tissue (e.g., at each pixel). By using the gradient-adjusted per-pixel phase to calculate per-pixel bulk modulus for the tissue of the treatment area 410, this bulk modulus information may be used, e.g., to determine or diagnose which areas or portions of tissue are cancerous or benign, and may also be used to diagnose what portions of tissue are associated with CNS (central nervous system) diseases, e.g., based on the bulk modulus of those pixels.

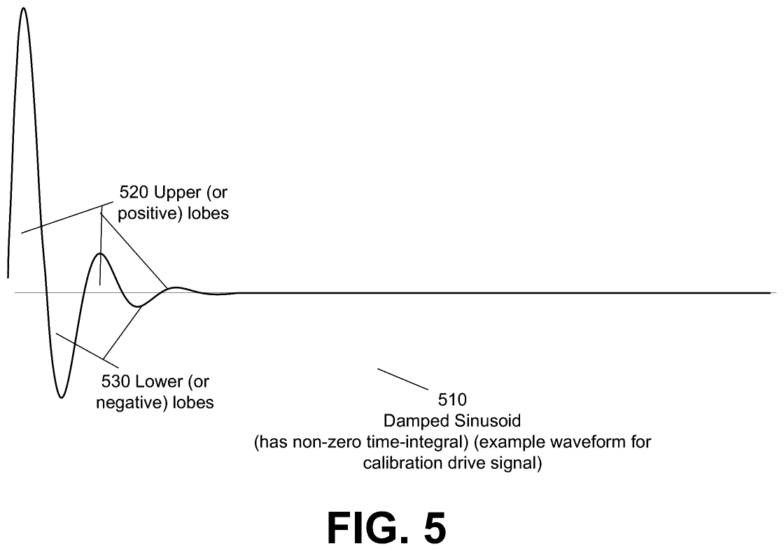

FIG. 5 is a diagram illustrating a damped sinusoid, which is an example waveform for the calibration drive signal used to drive the magnetic coil during calibration. Damped Sinusoid 510 may include upper (or positive) lobes 520 and lower (or negative) lobes 530. It can be seen that the sum of the upper lobes 520 (or area of upper lobes 520) is larger than the sum of the lower lobes 530 (or area of lower lobes 530). Thus, the damped sinusoid 510 has a non-zero time-integral (e.g., has a positive time-integral because area of the upper lobes 520 is greater than area of the lower lobes 530).

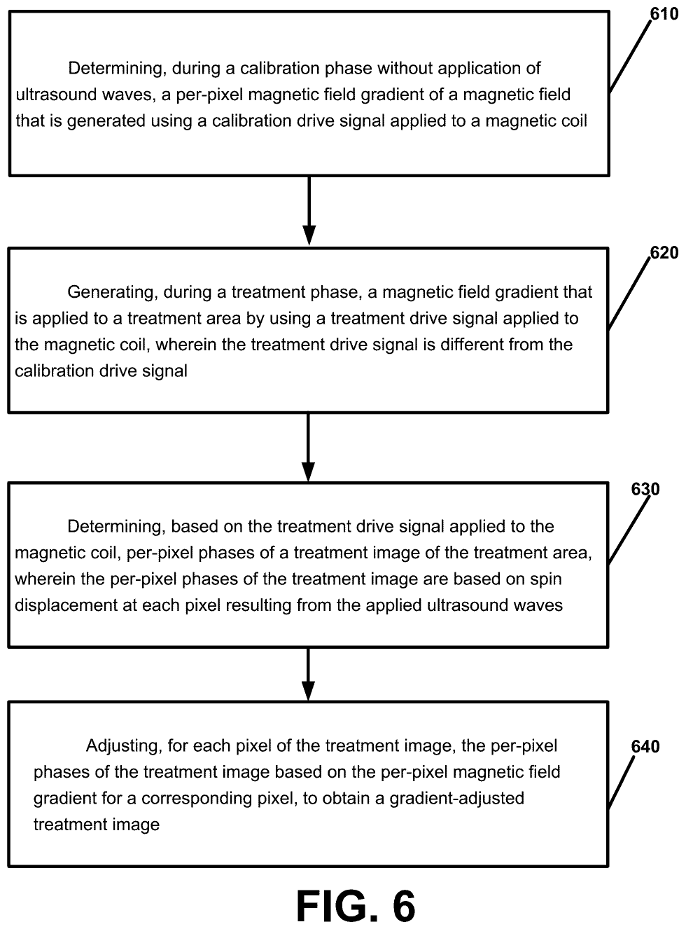

FIG. 6 is a flow chart illustrating operation of a system according to an example embodiment. The method of FIG. 6 illustrates a method of imaging based on ultrasound waves applied to a treatment area. Operation 610 includes determining, during a calibration phase without application of ultrasound waves, a per-pixel magnetic field gradient of a magnetic field that is generated using a calibration drive signal applied to a magnetic coil. Operation 620 includes generating, during a treatment phase, a magnetic field gradient that is applied to a treatment area by using a treatment drive signal applied to the magnetic coil, wherein the treatment drive signal is different from the calibration drive signal. Operation 630 includes determining, based on the treatment drive signal applied to the magnetic coil, per-pixel phases of a treatment image of the treatment area, wherein the per-pixel phases of the treatment image are based on spin displacement at each pixel resulting from the applied ultrasound waves. Operation 640 includes adjusting, for each pixel of the treatment image, the per-pixel phases of the treatment image based on the per-pixel magnetic field gradient for a corresponding pixel, to obtain a gradient-adjusted treatment image.

With respect to the method of FIG. 6, the calibration drive signal may have a waveform with a non-zero time-integral.

With respect to the method of FIG. 6, the treatment drive signal may have a waveform type that matches a waveform type of ultrasound waves that are applied to the treatment area during the treatment phase.

With respect to the method of FIG. 6, the gradient-adjusted treatment image may include, for each pixel in the gradient-adjusted treatment image, a gradient-adjusted per-pixel phase.

With respect to the method of FIG. 6, the waveform of the calibration drive signal, which has a non-zero time-integral, may be one of the following waveform types: an exponential waveform; a trapezoidal waveform; a square waveform; a ramp waveform; a triangle waveform; a damped sinusoidal waveform.

With respect to the method of FIG. 6, the waveform of the treatment drive signal and the waveform of the ultrasound waves may both be sinusoidal waveforms.

With respect to the method of FIG. 6, the determining a per-pixel magnetic gradient may include: applying, during the calibration phase without application of ultrasound waves, the calibration drive signal to the magnetic coil to generate the magnetic field during the calibration phase; and determining, during the calibration phase based on the applied calibration drive signal, the per-pixel magnetic field gradient as a gradient of the magnetic field at each pixel of a calibration image.

With respect to the method of FIG. 6, the adjusting may include: scaling, for each pixel of the treatment image, the per-pixel phases of the treatment image by or based on the per-pixel magnetic field gradient for the corresponding pixel, to obtain the gradient-adjusted treatment image.

With respect to the method of FIG. 6, the magnetic coil generates a non-linear magnetic field gradient over the treatment area (e.g., regardless of the type of drive signal applied to the magnetic coil).

With respect to the method of FIG. 6, the magnetic coil may generate a magnetic field having: an increasing or positive slope of the magnetic field gradient over a first region of the treatment area or a positive second derivative of the magnetic field over a first region of the treatment area; and a decreasing or negative slope of the magnetic field gradient over a second region of the treatment area, or a negative second derivative of the magnetic field over the second region of the treatment area.

With respect to the method of FIG. 6, the gradient-adjusted treatment image may include, for each pixel in the gradient-adjusted treatment image, a gradient-adjusted per-pixel phase, the method further including: determining, per-pixel, at least one acoustic parameter for the treatment area based on the gradient-adjusted per-pixel phase of the gradient-adjusted treatment image; wherein the at least one acoustic parameter includes at least one of the following: acoustic pressure amplitude; sound speed; or acoustic intensity.

With respect to the method of FIG. 6, the gradient-adjusted treatment image includes, for each pixel in the gradient-adjusted treatment image, a gradient-adjusted per-pixel phase, the method further including: determining, per-pixel, a bulk modulus for the treatment area based on the gradient-adjusted per-pixel phase of the gradient-adjusted treatment image.

An apparatus may include at least one processor; and at least one memory storing instructions that, when executed by the at least one processor, cause the apparatus at least to: determine, during a calibration phase without application of ultrasound waves, a per-pixel magnetic field gradient of a magnetic field that is generated using a calibration drive signal applied to a magnetic coil; generate, during a treatment phase, a magnetic field gradient that is applied to a treatment area by using a treatment drive signal applied to the magnetic coil, wherein the treatment drive signal is different from the calibration drive signal; determine, based on the treatment drive signal applied to the magnetic coil, per-pixel phases of a treatment image of the treatment area, wherein the per-pixel phases of the treatment image are based on spin displacement at each pixel resulting from the applied ultrasound waves; and adjust, for each pixel of the treatment image, the per-pixel phases of the treatment image based on the per-pixel magnetic field gradient for a corresponding pixel, to obtain a gradient-adjusted treatment image.

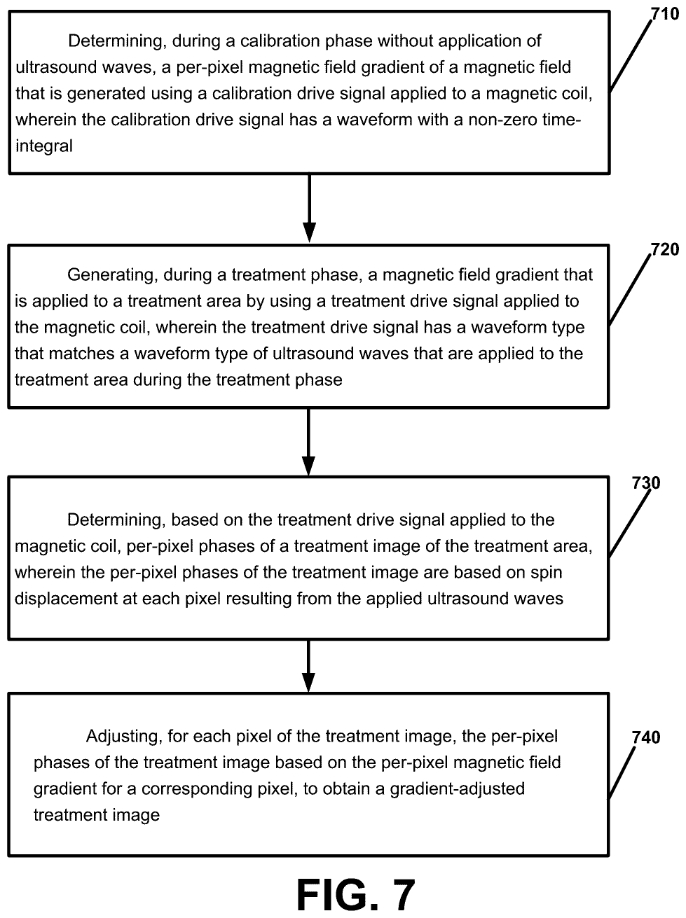

FIG. 7 is a flow chart illustrating operation of a system according to another example embodiment. Operation 710 may include determining, during a calibration phase without application of ultrasound waves, a per-pixel magnetic field gradient of a magnetic field that is generated using a calibration drive signal applied to a magnetic coil, wherein the calibration drive signal has a waveform with a non-zero time-integral. Operation 720 may include generating, during a treatment phase, a magnetic field gradient that is applied to a treatment area by using a treatment drive signal applied to the magnetic coil, wherein the treatment drive signal has a waveform type that matches a waveform type of ultrasound waves that are applied to the treatment area during the treatment phase. Operation 730 may include determining, based on the treatment drive signal applied to the magnetic coil, per-pixel phases of a treatment image of the treatment area, wherein the per-pixel phases of the treatment image are based on spin displacement at each pixel resulting from the applied ultrasound waves. And, operation 740 may include adjusting, for each pixel of the treatment image, the per-pixel phases of the treatment image based on the per-pixel magnetic field gradient for a corresponding pixel, to obtain a gradient-adjusted treatment image.

While certain features of the described embodiments have been illustrated as described herein, many modifications, substitutions, changes and equivalents will now occur to those skilled in the art. It is, therefore, to be understood that the appended claims are intended to cover all such modifications and changes as fall within the true spirit of the various embodiments.

What is claimed is:

1. A method of imaging based on ultrasound waves applied to a treatment area, comprising:
determining, during a calibration phase without application of ultrasound waves, a per-pixel magnetic field gradient of a magnetic field that is generated using a calibration drive signal applied to a magnetic coil;
generating, during a treatment phase, a magnetic field gradient that is applied to a treatment area by using a treatment drive signal applied to the magnetic coil, wherein the treatment drive signal is different from the calibration drive signal;
determining, based on the treatment drive signal applied to the magnetic coil, per-pixel phases of a treatment image of the treatment area, wherein the per-pixel phases of the treatment image are based on spin displacement at each pixel resulting from the applied ultrasound waves; and
adjusting, for each pixel of the treatment image, the per-pixel phases of the treatment image based on the per-pixel magnetic field gradient for a corresponding pixel, to obtain a gradient-adjusted treatment image.

2. The method of claim 1, wherein the calibration drive signal has a waveform with a non-zero time-integral.

3. The method of claim 1, wherein the treatment drive signal has a waveform type that matches a waveform type of ultrasound waves that are applied to the treatment area during the treatment phase.

4. The method of claim 1, wherein the gradient-adjusted treatment image includes, for each pixel in the gradient-adjusted treatment image, a gradient-adjusted per-pixel phase.

5. The method of claim 2, wherein the waveform of the calibration drive signal, which has a non-zero time-integral, is one of the following waveform types:
an exponential waveform;
a trapezoidal waveform;
a square waveform;
a ramp waveform;
a triangle waveform;
a damped sinusoidal waveform.

6. The method of claim 3, wherein the waveform of the treatment drive signal and the waveform of the ultrasound waves are both sinusoidal waveforms.

7. The method of claim 1, wherein the determining a per-pixel magnetic gradient comprises:
applying, during the calibration phase without application of ultrasound waves, the calibration drive signal to the magnetic coil to generate the magnetic field during the calibration phase; and
determining, during the calibration phase based on the applied calibration drive signal, the per-pixel magnetic field gradient as a gradient of the magnetic field at each pixel of a calibration image.

8. The method of claim 1, wherein the adjusting comprises:
scaling, for each pixel of the treatment image, the per-pixel phases of the treatment image by or based on the per-pixel magnetic field gradient for the corresponding pixel, to obtain the gradient-adjusted treatment image.

9. The method of claim 1, wherein the magnetic coil generates a non-linear magnetic field gradient over the treatment area.

10. The method of claim 1, wherein the magnetic coil generates a magnetic field having:
an increasing or positive slope of the magnetic field gradient over a first region of the treatment area or a positive second derivative of the magnetic field over a first region of the treatment area; and
a decreasing or negative slope of the magnetic field gradient over a second region of the treatment area, or a negative second derivative of the magnetic field over the second region of the treatment area.

11. The method of claim 1, wherein the gradient-adjusted treatment image includes, for each pixel in the gradient-adjusted treatment image, a gradient-adjusted per-pixel phase, the method further comprising:
determining, per-pixel, at least one acoustic parameter for the treatment area based on the gradient-adjusted per-pixel phase of the gradient-adjusted treatment image;
wherein the at least one acoustic parameter includes at least one of the following:
acoustic pressure amplitude;
sound speed; or
acoustic intensity.

12. The method of claim 1, wherein the gradient-adjusted treatment image includes, for each pixel in the gradient-adjusted treatment image, a gradient-adjusted per-pixel phase, the method further comprising:
determining, per-pixel, a bulk modulus for the treatment area based on the gradient-adjusted per-pixel phase of the gradient-adjusted treatment image.

13. An apparatus, comprising:
at least one processor; and
at least one memory storing instructions that, when executed by the at least one processor, cause the apparatus at least to:
determine, during a calibration phase without application of ultrasound waves, a per-pixel magnetic field gradient of a magnetic field that is generated using a calibration drive signal applied to a magnetic coil;
generate, during a treatment phase, a magnetic field gradient that is applied to a treatment area by using a treatment drive signal applied to the magnetic coil, wherein the treatment drive signal is different from the calibration drive signal;
determine, based on the treatment drive signal applied to the magnetic coil, per-pixel phases of a treatment image of the treatment area, wherein the per-pixel phases of the treatment image are based on spin displacement at each pixel resulting from the applied ultrasound waves; and
adjust, for each pixel of the treatment image, the per-pixel phases of the treatment image based on the per-pixel magnetic field gradient for a corresponding pixel, to obtain a gradient-adjusted treatment image.
